(19) United States
(12) Patent Application Publication
(10) Pub. No.: US 2010/0272601 A1
(43) Pub. Date: Oct. 28, 2010

(54) METHOD OF SELECTIVE TREATMENT OF MICROORGANISMS IN FOOD

(75) Inventor: Rafael Enamorado Solanes, Madrid (ES)

Correspondence Address: MERCHANT & GOULD PC, P.O. BOX 2903, MINNEAPOLIS, MN, 55402-0903, US

(73) Assignee: UNIVERSIDAD POLITECNICA DE MADRID, Madrid (ES)

(21) Appl. No.: 12/670,271

(22) PCT Filed: Jul. 21, 2008
(86) PCT No.: PCT/ES08/00512

§ 371 Date: Jul. 2, 2010

(30) Foreign Application Priority Data
Jul. 23, 2007 (ES) P200702053

(51) Int. Cl. A61L 2/08

(52) U.S. Cl. 422/22

(57) ABSTRACT

The invention refers to a method consisting of treating food through the application of low-frequency waves in resonance with each pathogen, said waves being non-ionizing radiations, which differ from dangerous high-frequency waves, which are ionizing.

This invention enables to obtain food without microbial contamination and without applying regular aggressive treatments. With this treatment a reduction of microbial contamination levels in food is obtained, acting on all kinds of microorganisms, both aerobic and anaerobic ones.

### FIELD OF THE INVENTION

The present invention refers to the technical field of the food industry.

### BACKGROUND OF THE INVENTION

Nowadays, food treated with regular procedures, either thermal or chemical, modify the nutritional components of treated food, which can produce health unbalances.

In the current state of the art, there exist pathogen control procedures, using electromagnetic waves; pathogen partial treatment procedures are described in U.S. 2007101943 for in-line control of poultry waste. In this procedure different frequency and intensity combinations are used, both radiofrequency and microwaves. The waves generated are a combination of pulsed waves, continuous waves and modulated waves.

U.S. 2007009377 describes a liquid treatment procedure through the transmission of electromagnetic radiations and its wavelength distribution through the container. The exposure of the container to electromagnetic radiations is carried out during time periods ranging from 0.1 to 30 minutes.

The exposition radiant energies are between 0.1 joule/cm and 10 joules/cm. The electromagnetic radiation is distributed in a wavelength range between 285 nanometers and 365 nanometers.

WO 2005089816 describes a pathogen reduction procedure in bottled liquids, where the electromagnetic radiation provided to the liquid through the container, partially transparent, is in the ultraviolet region spectrum, providing a photo-reactive effect for the polluted liquid.

WO02066080 claims a device which provides in vivo treatment for polluted surfaces, using a wide electromagnetic spectrum between 190 and 1200 nanometers. The device provides output signals of amplified scalar square waves.

The main novelty and interest of the present invention lies in the use of low-frequency waves which do not generate heat or radiation. They also have a great capacity of penetration in food, both solid and liquid, as a result of its action principle, which is the resonance with the microorganisms to be treated.

The present invention does not use the molecular agitation principles of microwaves (U.S. 2007101943), its corresponding temperature increase in all food and consequently generalized food deterioration.

The use of frequencies in the ultraviolet range (WO2005089816) has the limitations of its little penetration power in food.

U.S. 2007009377 and WO02066080 apply high frequencies which produce indiscriminate heat increases, affecting all components of food and surfaces treated.

### DETAILED DESCRIPTION OF THE ART

The present invention entails a novelty vis-à-vis current antimicrobial treatment procedures. The new industrial method differs from current methods in the non-aggressive application of food without thermally or chemically affecting the nutritional components of the food treated.

Besides, the food can be treated in all kinds of containers, open or closed, because the probes are placed outside the containers, and there is no interference in the transmission of low frequency waves.

The products obtained with the procedure described in the patent are food with a significant reduction of the microorganisms contained therein.

The present invention refers to a new pathogen treatment industrial method through the application of low frequency and low intensity waves, without producing ionization or denaturation of nutrients in the food treated.

The method is carried out through the generation of specific frequency waves in resonance with each pathogen. To that end, a scanning of frequencies ranging from 50 kHz and 10 MHz is carried out. These frequencies are used because the vibration frequencies of all microorganisms are contained within that range.

The voltage does not have much influence in the resonance and therefore, low voltages between 6 and 24 volts are used. It has been empirically proved that it is enough to have an application interval from 1 to 30 minutes following the regular methods thereto.

This resonance is a continued vibration of a system, in this case the microorganisms, which undergoes a stimulus and part of this energy passes thereto. If the energy supply is carried out at a determined frequency and close to the vibration of the microorganisms, the absorption rate is the highest possible rate and the oscillation amplitude increases, producing great instability in the microorganisms, thus causing their death.

The formula governing this kind of resonance is the base equation of this phenomenon: A=a₀/(w₀²−w²); wherein A=new amplitude for the application of the new frequency; w₀=new amplitude for the application with function generator; w=frequency of the system (microorganism);

a₀=value of the force applied; when w₀=is close to w, A increases until producing the complete destruction of the microorganism.

Additionally, to identify the specific frequencies for each pathogen, empirical tests can be carried out on samples inoculated with the microorganisms, through the use of an oscilloscope.

### AN EMBODIMENT OF THE INVENTION

The following is a description of the application of the method object of the patent on unpasteurized and unsterilized skimmed milk, providing an improved substrate to obtain cheese without saturated fat.

In order to carry out the treatment with the milk model, the process starts with unpasteurized and unsterilized skimmed milk. Before the treatment, a microbiological analysis is carried out to identify and quantify the microorganisms existing in the milk sample. Next, the milk is subjected to a frequency scanning with a frequency generator, contrasting the kind of wave, its frequency and voltage with an oscilloscope. Said frequency scanning in resonance with the microorganisms is carried out between 100 KHz and 10 MHz with a voltage between 6 and 24 volts, during an interval ranging from 1 to 30 minutes.

### INDUSTRIAL APPLICATION

This method can be applied to any kind of food. One of the first practical applications of this invention is the treatment of unpasteurized and unsterilized skimmed milk, for later obtaining cheese without saturated fat.

1. Method of selective treatment of microorganisms in food, comprising generating specific frequency waves in resonance with each pathogen through a scanning of waves which frequencies range from 50 kHz to 10 MHz with a voltage between 6 and 24, and during an interval from 1 to 30 minutes, treated through probes, without causing ionization in the food.

2. Method of selective treatment of microorganisms in food according to claim 1, wherein before the generation of frequencies a microbiological analysis can be carried out to quantify the microorganisms existing in the food and verify their state after performing the specific frequency wave scanning in resonance with each pathogen.

3. Method of selective treatment of microorganisms in food according to claim 1, wherein the food to be treated is located in open or closed containers, receiving the frequencies through probes located outside the container.
